SEQUENCE LISTING (85 sequences)

SEQ ID NO: 1 - RNA, 22 nt, Homo sapiens
ugagguaguagguuguauaguu

SEQ ID NO: 2 - DNA, 77 bp, Artificial Sequence
  Synthetic template DNA for detection of let-7a miRNA
  misc_feature (77)..(77): 3′ terminal nucleotide is dideoxy C analog
ctaatacgactcactatagggagagctgaaatcacaaatacaacgaatcgagtaaactatacaacctactacctcac

SEQ ID NO: 3 - RNA, 22 nt, Homo sapiens
ugagguaguagguuguaugguu

SEQ ID NO: 4 - DNA, 65 bp, Artificial Sequence
  Synthetic DNA template for detection of let-7c miRNA
  misc_feature (65)..(65): 3′ terminal nucleotide is dideoxy C analog
ctaatacgactcactatagggagagcgataaattagaattcgaaccatacaacctactacctcac

SEQ ID NO: 5 - RNA, 76 nt, Artificial Sequence
  Synthetic RNA template for detection of let-7a miRNA
uauuccagacucaccuuauacacagcugaaaucacaaauacaacgaaucgaguaaacuauucaaccuacuaccuca

SEQ ID NO: 6 - DNA, 22 bp, Artificial Sequence
  PCR primer specific for spacer sequence of DNA template
ttactcgattgcttgtatttgt

SEQ ID NO: 7 - DNA, 19 bp, Artificial Sequence
  PCR primer for amplifying extended miRNA
ttccagactcaccttatac

SEQ ID NO: 8 - RNA, 83 nt, Homo sapiens
gagaaucaucucucccagauaauggcacucucaaacaaguuuccaaauuguuugaaaggcuauuucuuggucagaugacucuc

SEQ ID NO: 9 - RNA, 84 nt, Homo sapiens
guggcagcuugguggucguaugugugacgccauuuacuugaaccuuuaggagugacaucacauauacggcagcuaaacugcuac

SEQ ID NO: 10 - RNA, 128 nt, Homo sapiens
uggaggccuugcugguuuggaaaguucauuguucgacaccauggaucuccaggugggucaaguuuagagaugcaccaaccuggaggacuccaugcuguugagcuguucacaagcagcggacacuucca

SEQ ID NO: 11 - RNA, 84 nt, Homo sapiens
uugacuuagcuggguaguggggaacccuuccaugaggaguagaacacuccuuaugcaagauucccuucuaccuggcuggguugg

SEQ ID NO: 12 - RNA, 87 nt, Homo sapiens
caauagacacccaucgugucuuuugcucugcagucaguaaauauuuuuuugugaauguguagcaaaagacagaauggugguccauug

SEQ ID NO: 13 - RNA, 87 nt, Homo sapiens; identical to SEQ ID NO: 12

SEQ ID NO: 14 - RNA, 73 nt, Homo sapiens
ccuggcacugagaacugaauuccauaggcugugagcucuagcaaugcccuguggacucaguucuggugcccgg

SEQ ID NO: 15 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 16 - RNA, 116 nt, Homo sapiens
caacuacagccacuacuacaggaccaucgaggaccugcgggacaagauucuuggugccaccauugagaacgccaggauuguccugcagaucaacaaugcucaacuggcugcagaug

SEQ ID NO: 17 - RNA, 89 nt, Homo sapiens
cuggccuccagggcuuuguacaugguaggcuuucauucauucguuugcacauucggugaaggucuacugugugccaggcccugugccag

SEQ ID NO: 18 - RNA, 94 nt, Homo sapiens
ugacuccuccaggucuuggaguaggucauuggguggauccucuauuuccuuacgugggccacuggauggcuccuccaugucuuggaguagauca

SEQ ID NO: 19 - RNA, 81 nt, Homo sapiens
gauacucgaaggagagguuguccguguugucuucucuuuauuuaugaugaaacauacacgggaaaccucuuuuuuaguauc

SEQ ID NO: 20 - RNA, 82 nt, Homo sapiens
ugguaccugaaaagaaguugcccauguuauuuucgcuuuauaugugacgaaacaaacauggugcacuucuuuuucgguauca

SEQ ID NO: 21 - RNA, 102 nt, Homo sapiens
cccaagucagguacucgaauggagguuguccaugguguguucauuuuauuuaugaugaguauuacauggccaaucuccuuucgguacucaauucuucuuggg

SEQ ID NO: 22 - RNA, 83 nt, Homo sapiens
guggucucagaaucgggguuuugagggcgagaugaguuuauguuuuauccaacuggcccucaaagucccgcuuuuggggucau

SEQ ID NO: 23 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 24 - RNA, 137 nt, Homo sapiens
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 25 - RNA, 84 nt, Homo sapiens
ucucagucuguggcacucagccuugagggcacuuucuggugccagaaugaaagugcugucauagcugagguccaaugacugagg

SEQ ID NO: 26 - RNA, 98 nt, Homo sapiens
gguacuucucagucuguggcacucagccuugagggcacuuucuggugccagaaugaaagugcugucauagcugagguccaaugacugaggcgagcacc

SEQ ID NO: 27 - RNA, 124 nt, Homo sapiens
aacccuccuugggaagugaagcucaggcugugauuucaagccagggggcguuuuucuauaacuggaugaaaagcaccuccagagcuugaagcucacaguuugagagcaaucgucuaaggaaguu

SEQ ID NO: 28 - RNA, 87 nt, Homo sapiens
ucuccugcugugacccucaagauggaagcaguuucuguugucugaaaggaaagaaagugcuuccuuuuugaggguuacuguuugaga

SEQ ID NO: 29 - RNA, 83 nt, Homo sapiens
ucucaugcagucauucuccaaaagaaagcacuuucuguugucugaaagcagagugccuucuuuuggagcguuacuguuugaga

SEQ ID NO: 30 - RNA, 84 nt, Homo sapiens
ucucaugcagucauucuccaaaagggagcacuuucuguuugaaagaaaacaaagugccuccuuuuagaguguuacuguuugaga

SEQ ID NO: 31 - RNA, 87 nt, Homo sapiens
ucucaggcugugacccucuaaagggaagcgcuuucuguggucagaaagaaaagcaagugcuuccuuuuagaggguuaccguuuggga

SEQ ID NO: 32 - RNA, 83 nt, Homo sapiens; identical to SEQ ID NO: 29

SEQ ID NO: 33 - RNA, 87 nt, Homo sapiens
ucucagccugugacccucuagagggaagcgcuuucuguugucugaaagaaaagaaagugcaucuuuuuagaggauuacaguuugaga

SEQ ID NO: 34 - RNA, 85 nt, Homo sapiens
cucaggcugugacccuccagagggaaguacuuucuguugucugagagaaaagaaagugcuucccuuuggacuguuucgguuugag

SEQ ID NO: 35 - RNA, 83 nt, Homo sapiens
ucaggcugugacccucuugagggaagcacuuucuguugucugaaagaagagaaagugcuuccuuuuagaggcuuacugucuga

SEQ ID NO: 36 - RNA, 81 nt, Homo sapiens
caugcugugacccucuagagggaagcgcuuucuguugucugaaagaaaagaaagugcauccuuuuagagguuuacuguuug

SEQ ID NO: 37 - RNA, 85 nt, Homo sapiens
cucaagcugugacucuccagagggaugcacuuucucuuaugugaaaaaaaagaaggcgcuucccuuuagagcguuacgguuuggg

SEQ ID NO: 38 - RNA, 87 nt, Homo sapiens
ucucaugcugugacccucuagagggaagcgcuuucuguugucugaaagaaaagaacgcgcuucccuauagaggguuacccuuugaga

SEQ ID NO: 39 - RNA, 87 nt, Homo sapiens
ucucaugcugugacccucuagagggaagcacuuucucuugucuaaaagaaaagaaagcgcuucucuuuagaggauuacucuuugaga

SEQ ID NO: 40 - RNA, 61 nt, Homo sapiens
cccucuacagggaagcgcuuucuguugucugaaagaaaagaaagugcuuccuuuuagaggg

SEQ ID NO: 41 - RNA, 83 nt, Homo sapiens
ucaugcuguggcccuccagagggaagcgcuuucuguugucugaaagaaaacaaagcgcuccccuuuagagguuuacgguuuga

SEQ ID NO: 42 - RNA, 85 nt, Homo sapiens
cucaggcugugacccucuagagggaagcacuuucuguugcuugaaagaagagaaagcgcuuccuuuuagaggauuacucuuugag

SEQ ID NO: 43 - RNA, 87 nt, Homo sapiens
ucucaggcugucguccucuagagggaagcacuuucuguugucugaaagaaaagaaagugcuuccuuuuagaggguuaccguuugaga

SEQ ID NO: 44 - RNA, 101 nt, Homo sapiens
gcgagaagaucucaugcugugacucucuggagggaagcacuuucuguugucugaaagaaaacaaagcgcuucucuuuagaguguuacgguuugagaaaagc

SEQ ID NO: 45 - RNA, 87 nt, Homo sapiens
ucucaugcugugacccuacaaagggaagcacuuucucuuguccaaaggaaaagaaggcgcuucccuuuggaguguuacgguuugaga

SEQ ID NO: 46 - RNA, 87 nt, Homo sapiens
ucucaggcagugacccucuagauggaagcacugucuguuguauaaaagaaaagaucgugcaucccuuuagaguguuacuguuugaga

SEQ ID NO: 47 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 48 - RNA, 87 nt, Homo sapiens
ucucgggcugugacucuccaaagggaagaauuuucucuugucuaaaagaaaagaacgcacuucccuuuagaguguuaccgugugaga

SEQ ID NO: 49 - RNA, 87 nt, Homo sapiens
ucucaagcugugagucuacaaagggaagcccuuucuguugucuaaaagaaaagaaagugcuucucuuugguggguuacgguuugaga

SEQ ID NO: 50 - RNA, 67 nt, Homo sapiens
gugacccucuagauggaagcacugucuguugucuaagaaaagaucgugcaucccuuuagaguguuac

SEQ ID NO: 51 - RNA, 90 nt, Homo sapiens
ucccaugcugugacccucuagaggaagcacuuucuguuuguugucugagaaaaaacaaagugcuucccuuuagaguguuaccguuuggga

SEQ ID NO: 52 - RNA, 85 nt, Homo sapiens
ucucaugaugugaccaucuggagguaagaagcacuuuguguuuugugaaagaaagugcuuccuuucagaggguuacucuuugaga

SEQ ID NO: 53 - RNA, 65 nt, Homo sapiens
gugacccucuagagggaagcacuuucuguugaaagaaaagaacaugcauccuuucagaggguuac

SEQ ID NO: 54 - RNA, 88 nt, Homo sapiens
ucucaggcugugacccucuagagggaagcgcuuucuguuggcuaaaagaaaagaaagcgcuucccuucagaguguuaacgcuuugaga

SEQ ID NO: 55 - RNA, 85 nt, Homo sapiens
ucucaagcugugacugcaaagggaagcccuuucuguugucugaaagaagagaaagcgcuucccuuugcuggauuacgguuugaga

SEQ ID NO: 56 - RNA, 87 nt, Homo sapiens
ucccaugcugugacccucuagagggaagcacuuucuguugucugaaagaaaccaaagcgcuucccuuuggagcguuacgguuugaga

SEQ ID NO: 57 - RNA, 90 nt, Homo sapiens
ucucaggcugugaccaucuggagguaagaagcacuuucuguuuugugaaagaaaagaaagugcuuccuuucagaggguuacucuuugaga

SEQ ID NO: 58 - RNA, 87 nt, Homo sapiens
ucucaagcugugggucugcaaagggaagcccuuucuguugucuaaaagaagagaaagcgcuucccuuugcuggauuacgguuugaga

SEQ ID NO: 59 - RNA, 95 nt, Homo sapiens
gaagaucucaggcagugacccucuagauggaagcacugucuguugucuaagaaaagaucgugcauccuuuuagaguguuacuguuugagaaaauc

SEQ ID NO: 60 - RNA, 88 nt, Homo sapiens
ucccaugcugugacccucuagaggaagcacuuucuguuuguugucugagaaaaaacaaagugcuucccuuuagaguuacuguuuggga

SEQ ID NO: 61 - RNA, 87 nt, Homo sapiens
ucucaggcugugacccuccaaagggaagaacuuucuguugucuaaaagaaaagaacgcacuucccuuuagaguguuaccgugugaga

SEQ ID NO: 62 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 63 - RNA, 85 nt, Homo sapiens
cucaggcugugacacucuagagggaagcgcuuucuguugucugaaagaaaggaaagugcauccuuuuagaguguuacuguuugag

SEQ ID NO: 64 - RNA, 85 nt, Homo sapiens
ucucaagcugugacugcaaagggaagcccuuucuguugucuaaaagaaaagaaagugcuucccuuuggugaauuacgguuugaga

SEQ ID NO: 65 - RNA, 90 nt, Homo sapiens
ucucaggcugugaccuucucgaggaaagaagcacuuucuguugucugaaagaaaagaaagugcuuccuuucagaggguuacgguuugaga

SEQ ID NO: 66 - RNA, 90 nt, Homo sapiens
ucucagguugugaccuucucgaggaaagaagcacuuucuguugucugaaagaaaagaaagugcuuccuuucagaggguuacgguuugaga

SEQ ID NO: 67 - RNA, 87 nt, Homo sapiens
ucucaggcugugucccucuacagggaagcgcuuucuguugucugaaagaaaggaaagugcauccuuuuagaguguuacuguuugaga

SEQ ID NO: 68 - RNA, 122 nt, Homo sapiens
gcccuguccccugugccuugggcgggcggcuguuaagacuugcagugauguuuaacuccucuccacgugaacaucacagcaagucugugcugcuucccgucccuacgcugccugggcagggu

SEQ ID NO: 69 - RNA, 84 nt, Homo sapiens
gcucccccucucuaauccuugcuaccugggugagagugcugucugaaugcaaugcaccugggcaaggauucugagagcgagagc

SEQ ID NO: 70 - RNA, 84 nt, Homo sapiens
gcucuuccucucuaauccuuugucccugggugagagugcuuucugaaugcaaugcacccgggcaaggauucugagagggugagc

SEQ ID NO: 71 - RNA, 55 nt, Homo sapiens
auccuugcuaucugggugcuagugcuggcucaaugcaaugcaccugggcaaggau

SEQ ID NO: 72 - RNA, 100 nt, Homo sapiens
ccaaagaaagaugcuaaacuauuuuugcgauguguuccuaauauguaauauaaauguauuggggacauuuugcauucauaguuuuguaucaauaauaugg

SEQ ID NO: 73 - RNA, 71 nt, Homo sapiens
ugcccuagcagcgggaacaguucugcagugagcgaucggugcucugggguauuguuuccgcugccagggua

SEQ ID NO: 74 - RNA, 83 nt, Homo sapiens
gcugcuguugggagacccuggucugcacucuaucuguauucuuacugaagggagugcagggcaggguuucccauacagagggc

SEQ ID NO: 75 - RNA, 84 nt, Homo sapiens
gaugcacccagugggggagccaggaaguauugauguuucugccaguuuagcgucaacacuugcugguuuccucucuggagcauc

SEQ ID NO: 76 - RNA, 129 nt, Homo sapiens
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 77 - RNA, 127 nt, Homo sapiens
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 78 - RNA, 124 nt, Homo sapiens
gccaccaccaucagccauacuauguguagugccuuauucaggaagguguuacuuaauagauuaauauuuguaaggcacccuucugaguagaguaaugugcaacauggacaacauuugugguggc

SEQ ID NO: 79 - RNA, 94 nt, Homo sapiens
gugcuguguguagugcuucacuucaagaagugccaugcaugugucuagaaauauguuuugcaccuuuuggagugaaauaaugcacaacagauac

SEQ ID NO: 80 - RNA, 115 nt, Homo sapiens
ccaccuucagcugaguguagugcccuacuccagagggcgucacucauguaaacuaaaacaugauuguagccuuuuggaguagaguaauacacaucacguaacgcauauuuggugg

SEQ ID NO: 81 - RNA, 94 nt, Homo sapiens
caugcugugugugguacccuacugcagacaguggcaaucauguauaauuaaaaaugauugguacgucuguggguagaguacugcaugacacaug

SEQ ID NO: 82 - RNA, 74 nt, Homo sapiens
gugguguccuacucaggagaguggcaaucacauguaauuaggugugauugaaaccucuaagaguggaguaacac

SEQ ID NO: 83 - RNA, 58 nt, Homo sapiens
cuacucuggagagugacaaucauguauaauuaaauuugauugacacuucugugaguag

SEQ ID NO: 84 - RNA, 58 nt, Homo sapiens
cuacucuggagagugacaaucauguauaacuaaauuugauugacacuucugugaguag

SEQ ID NO: 85 - RNA, 58 nt, Homo sapiens; identical to SEQ ID NO: 84

(19) United States
(12) Patent Application Publication
(10) Pub. No.: US 2007/0077582 A1
(43) Pub. Date: Apr. 5, 2007

(54) Method for quantitative detection of short RNA molecules

(75) Inventor: Vladimir I. Slepnev, Newton, MA (US)

Correspondence Address: PALMER & DODGE, LLP;KATHLEEN M. WILLIAMS, 111 HUNTINGTON AVENUE, BOSTON, MA, 02199, US

(73) Assignee: Primera BioSystems, Inc., Mansfield, MA (US)

(21) Appl. No.: 11/523,687

(22) Filed: Sep. 18, 2006

Related U.S. Application Data
(60) Provisional application No. 60/717,638, filed on Sep. 16, 2005.

(51) Int. Cl. C12Q 1/68; C12P 19/34

(52) U.S. Cl. 435/6; 435/91.2

(57) ABSTRACT

Described herein are approaches to the detection and quantitation of short RNAs in a biological sample. The methods permit the detection and quantitation of individual species of short RNA in a nucleic acid sample, both singly and in a multiplex format that permits the determination of expression levels for two or more target short RNAs in a single reaction.

### RELATED APPLICATIONS

This application claims priority to U.S. provisional application 60/717,638, filed Sep. 16, 2005. The entire teachings of the above application is incorporated herein by reference.

### FIELD OF THE INVENTION

The present invention provides for methods of identifying and quantifying of short RNA molecules including, for example, micro RNA (miRNA), short interfering RNA (siRNA), short hairpin RNA (shRNA).

### BACKGROUND OF THE INVENTION

Short RNA molecules (less than 50 bases) play an important role regulating gene expression in variety of cells. Naturally occurring microRNAs (miRNAs) are a class of small noncoding RNAs that regulate a wide range of cellular processes (Eddy, Nat Rev Genet, 2001, 2, 919-929; Kawasaki and Taira, Nature, 2003, 423, 838-842). MiRNAs are ubiquitous, having been identified in plants and animals ( John B, Enright A J, Aravin A, Tuschl T, Sander C, et al. (2004) Human MicroRNA Targets. PLoS Biol 2(11): e363, citing (Lee et al. 1993; Reinhart et al. 2000, 2002; Lagos-Quintana et al. 2001, 2002, 2003; Lau et al. 2001; Lee and Ambros 2001; Llave et al. 2002a; Mette et al. 2002; Mourelatos et al. 2002; Park et al. 2002; Ambros et al. 2003b; Aravin et al. 2003; Brennecke et al. 2003; Dostie et al. 2003; Grad et al. 2003; Houbaviy et al. 2003; Lai et al. 2003; Lim et al. 2003a, 2003b; Palatnik et al. 2003). More than 300 different miRNAs have been identified in humans according to the miRBase (http://microma.sanger.ac.uk/) and The microRNA Registry, (Griffiths-Jones S. NAR, 2004, 32, Database Issue, D109-D111) and Ambros et al. (Curr Biol, 2003, 13, 807-818). MiRNAs have also been identified in the Epstein Barr virus, ( John B. et al., supra, citing Pfeffer et al. 2004,) and are differentially expressed in developmental stages, cell types, and tissues (Lee and Ambros 2001; Lagos-Quintana et al. 2002; Sempere et al. 2004). In particular, differential expression has been observed in mammalian organs (Lagos-Quintana et al. 2002; Krichevsky et al. 2003; Sempere et al. 2004) and embryonic stem cells (Houbaviy et al. 2003). A list of known miRNAs can be obtained by accessing FTP director/pub/mirbase/sequences/CURRENT/ at ftp.sanger.ac.uk. A listing of miRNAs available as of Sep. 13, 2005 is described herein, (see Appendix 1).

MiRNAs are encoded by several hundred novel genes, which encode transcripts containing short double-stranded RNA hairpins. MiRNAs are transcribed as longer precursors, termed pre-miRNAs (John B. et al., supra, citing Lee et al. 2002), which are usually 50 to 80 nucleotides in length, and which are sometimes found in clusters and frequently found in introns, (John B. et al., supra) Upon transcription, miRNAs undergo nuclear cleavage by the RNase III endonuclease Drosha, producing the 60-70-nt stem-loop precursor miRNA (pre-miRNA) with a 5′ phosphate and a 2-nt 3 overhang (John B. et al., supra, citing Lee et al. 2003). The pre-miRNAs are cleaved by Dicer about two helical turns away from the ends of the pre-miRNA stem loop, producing double-stranded RNA with strands that are approximately the same length (21 to 24 nucleotides), and possess the characteristic 5′-phosphate and 3′-hydroxyl termini. One of the strands of this short-lived intermediate accumulates as the mature miRNA and is subsequently incorporated into a ribonucleoprotein complex, the miRNP, which is similar, if not identical to the RISC (John B. et al., supra, citing Ambros et al., RNA, 2003, 9, 277-279, Bartel and Bartel, Plant Physiol, 2003, 132, 709-717, Shi, Trends Genet, 2003, 19,9-12).

MiRNAs interact with target niRNAs at specific sites to induce cleavage of the message or inhibit translation. (John et al. supra). Synthetic miRNAs have been made and used to analyze the mechanisms of miRNAs (McManus et al., RNA, 2002, 8, 842-850). Naturally occurring miRNAs are also believed to be involved in the regulation of a wide range of cellular processes, including development and oncogenesis. Deregulation of miRNA expression may contribute to inappropriate survival that occurs in oncogenesis (Xu et al., Curr Biol, 2003, 13, 790-795).

The challenge of quantitative detection of miRNAs outlines the problems common for quantitation of all short RNA sequences. Routine methods for RNA quantitation are based on first, reverse transcription of RNA into cDNA, and then detection of complementary cDNA using various methods for amplification of signal or cDNA molecules using for example the Reverse-Transcription—PCR amplification approach. For short RNA sequences, reverse transcription creates very short cDNA molecules, making the design of two non-overlaping DNA primers extremely challenging. Different approaches have been suggested to address this problem. One approach uses novel stem-loop RT followed by TaqMan PCR analysis (Chen et al. Nucleic Acids Res. 2005;33(20), el79. This method includes reverse transcription at low temperature. Another approach is to use a composite primer for reverse transcription which includes a gene-specific portion and a tail sequence used for PCR amplification (Raymond et al. RNA. 2005 Nov.; 11(l1):1737-44).

### SUMMARY OF THE INVENTION

Described herein are approaches to the identification, detection and quantitation of short RNAs in a biological sample. These approaches provide a means of identifying and quantitating a short RNAs by detecting and quantifying a product which is generated by extending the short RNA sequencee. The approaches described herein provide the advantage of not only permitting the specific detection and quantitation of an individual species of short RNA in a nucleic acid sample, but also provide for a multiplex format that permits the determination of expression levels for two or more short RNAs in a single reaction.

Described herein is an alternative approach to the detection and quantitation of short RNA sequences based on using the target short RNA as a primer for extension by DNA polymerase on a specific oligonucleotide template. This specific oligonucleotide sequence is preferably longer than the target short RNA sequence and contains at its 3′-end a sequence complementary to target short RNA and a spacer sequence adjacent to that complimentary sequence, which is used in subsequent signal amplification.

In one embodiment the specific oligonucleotide template is an RNA molecule. The extension of short RNA using reverse transcriptase creates a DNA copy of the specific oligonucleotide template. This DNA copy can be further detected by PCR amplification using primers directed to the spacer region.

In another embodiment the specific oligonucleotide template is a DNA molecule. The sequence of the template contains at its 3′-end a sequence complimentary to target short RNA sequence, a spacer and a sequence encoding an RNA polymerase promoter. The extension of annealed short RNA creates a double-stranded DNA which contains a functional promoter for RNA polymerase. An RNA polymerase-mediated transcription creates multiple copies of RNAs complimentary to the spacer region which can be detected directly or further amplified in an RT-PCR reaction using primers directed to the spacer sequence. Preferably,the latter step is preceeded by a treatment with heat-sensitive DNAse to eliminate unused copies of the specific oligonucleotide sequence.

Given that naturally occurring miRNAs play a role in regulating a wide range of physiological conditions and processes, the novel methods of identifying miRNAs disclosed herein can be applied to methods involving the diagnosis, prognosis and/or staging of miRNA associated diseases, disorders and conditions involving infections from bacteria, viruses and fungi, as well as miRNA associated diseases/disorders and conditions resulting from aberrant gene expression, including dis-regulation of genes involved in autoimmunity, cancer, inflammation and apoptosis, as well as in methods involving development and homeostasis in individuals.

### DEFINITIONS

As used herein, “short RNA molecule” means an RNA molecule containing less than 50 nucleobases in length and more than 5 bases in length. “Short RNA” includes but is not limited to biologically active RNA molecules such as microRNA (miRNAs), short interfering RNAs (siRNAs), short hairpin RNAs (shRNAs) or RNA species derived from aforementioned classes of RNAs by metabolic processes.

As used herein, “random” means a nucleotide sequence, wherein each nucleotide of the sequence has an equal probability of occurring.

As used herein, a “spacer of defined length” means a nucleotide sequence which consists of a polynucleotide sequence containing a known number of nucleotides. The number of nucleotides, or analogues thereof, in the spacer of defined length can range from at least 1 nucleotide, or analogue thereof, up to 2, 3, 4, 5, 6, 7, 8, 9, 10, 11, 12, 13, 14, 15, 16, 17, 18, 19, 20, 21, 22, 23, 24, 25, 30, 40, 50, 75, 100, 200, 300, 400, 500, 750, 1000, or up to approximately 1250 nucleotides or analogues thereof.

As used herein, a “spacer” is a sequence that is not associated with a given target short RNA in nature. Most often, for example, a spacer will be a heterologous sequence selected by the user to have a known sequence that is appended to sequence complementary to the target short RNA in a given template molecule. A “spacer” as the term is used herein is distinct from an RNA polymerase promoter sequence or its complement.

As used herein, the terms “nucleic acid”, “polynucleotide” and “oligonucleotide” generally refer to any polyribonucleotide or poly-deoxyribonucleotide, which may be unmodified RNA or DNA or modified RNA or DNA. “Polynucleotides” include, without limitation, single- and double-stranded polynucleotides. The term “polynucleotides” as it is used herein embraces chemically, enzymatically or metabolically modified forms of polynucleotides, as well as the chemical forms of DNA and RNA characteristic of viruses and cells, including for example, simple and complex cells. A polynucleotide useful for the methods described herein may be an isolated or purified polynucleotide or it may be an amplified polynucleotide in an amplification reaction, or a transcribed product in an in vitro transcription method.

As used herein, the terms “nucleic acid”, “polynucleotide” and “oligonucleotide” also encompass primers and probes, as well as oligonucleotide fragments, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

As used herein, “extending” refers to any in vitro method for making a new strand of polynucleotide or elongating an existing polynucleotide (i.e., DNA or short RNA) in a template dependent manner. The act of extending according to the methods described herein, can include amplification, which increases the number of copies of a polynucleotide template sequence with the use of a polymerase. Extending a polynucleotide results in the incorporation of nucleotides into a polynucleotide (i.e., including a polymerse recognition site, or a spacer of defined length), thereby forming an extended polynucleotide molecule complementary to the polynucleotide template. The extended polynucleotide molecule can be used as a template for PCR amplification or as a template to transcribe polynucleotide molecules, which are complementary to a short RNA target and which contain a tag of variable length. Optionally the transcription can be performed in the presence of labeled nucleotides or ribonucleotides, facilitating detection and quantitation.

As used herein, an “extended short RNA product” is the product of an extension reaction that adds nucleotides to a short RNA target molecule. The extended short RNA product can have deoxyribonucleotide bases or ribonucleotide bases or both, or modified labeled or unnatural bases, depending upon what bases are provided by the user for the extension reaction.

As used herein, the term “lacks 5′-nuclease activity” means that a given enzyme, e.g., a polymerase enzyme, substantially lacks 5′ exonuclease activity. An enzyme “substantially lacks” 5′ exonuclease activity when it has either no exonuclease activity or when it has less than 5% of the exonuclease activity of Vent™ polymerase. Thus, for example, Vent Exo-™ substantially lacks 5′ nuclease activity, as would another enzyme with less than 5% of the 5′ exonuclease activity of Vent™ polymerase.

As used herein, reference to a “size distinguishable by capillary electrophoresis” means a difference of at least one nucleotide, but preferably at least 5 nucleotides or more.

As used herein, the term “sample” refers to a biological material which is isolated from its natural environment and contains a polynucleotide. A “sample” according to the methods described herein may be a tissue or cell extract or it may contain purified or isolated polynucleotide(s).

As used herein, an “oligonucleotide primer” refers to a polynucleotide molecule (i.e., DNA, RNA or a combination thereof) capable of annealing to a polynucleotide template and providing a 3′ end to produce an extension product which is complementary to the polynucleotide template. The conditions for initiation and extension usually include the presence of four different deoxyribonucleoside triphosphates (dNTPs) and a polymerization-inducing agent such as a DNA polymerase or a reverse transcriptase activity, in a suitable buffer (“buffer” includes substituents which are cofactors, or which affect pH, ionic strength, etc.) and at a suitable temperature. The primer as described herein may be single- or double-stranded. The primer is preferably single-stranded for maximum efficiency in amplification. “Primers” useful in the methods described herein are less than or equal to 100 nucleotides in length, e.g., less than or equal to 90, or 80, or 70, or 60, or 50, or 40, or 30, or 20, or 15, but preferably longer than 10 nucleotides in length. In the case of the methods described herein, it is preferable that the primer hybridize to at least the 3′ end of an short RNA target.

As used herein, the term “oligonucleotide template” refers to a polynucleotide molecule (e.g., DNA, RNA or a combination thereof) capable of annealing to a short RNA target so as to permit polymerase extension of the short RNA target to form an extension product complementary to the oligonucleotide template. An oligonucleotide template as used herein has a sequence at or near its 3′ end that is complementary to a short RNA target, e.g., an miRNA target. The sequence complementary to to the short RNA target is sufficiently long, considering variables described herein, to specifically and stably anneal to the target short RNA such that the short RNA can be extended by a polymerase under conditions suitable and sufficient for activity of the polymerase. An oligonucleotide template further has a spacer sequence as described herein, which provides a known sequence of known length for each different template. The spacer sequence on each template provides a means for unambiguously determining the presence of extension or amplification products comprising that spacer sequence or its complement, and therefore the presence of sequence complementary to the template in a given sample.

As used herein, “label” or “detectable label” refers to any moiety or molecule which can be used to provide a detectable (preferably quantifiable) signal. A “labeled nucleotide” (e.g., a NTP or dNTP), or “labeled polynucleotide”, is one linked to a detectable label. The term “linked” encompasses covalently and non-covalently bonded, e.g., by hydrogen, ionic, or Van der Waals bonds. Such bonds may be formed between at least two of the same or different atoms or ions as a result of redistribution of electron densities of those atoms or ions. Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. A nucleotide useful in the methods described herein can be labeled so that the transcribed product may incorporate the labeled nucleotide and becomes detectable. A fluorescent dye is a preferred label according to the methods described herein. Suitable fluorescent dyes include fluorochromes such as Cy5, Cy3, rhodamine and derivatives (such as Texas Red), fluorescein and derivatives (such as 5-bromomethyl fluorescein), Lucifer Yellow, IAEDANS, 7-Me₂N-coumarin-4-acetate, 7-OH-4-CH₃-coumarin-3-acetate, 7-NH₂-4-CH₃-coumarin-3-acetate (AMCA), monobromobimane, pyrene trisulfonates, such as Cascade Blue, and monobromorimethyl-ammoniobimane (see for example, DeLuca, Immunofluorescence Analysis, in Antibody As a Tool, Marchalonis et al., eds., John Wiley & Sons, Ltd., (1982), which is incorporated herein by reference).

It is intended that the term “labeled nucleotide”, as used herein, also encompasses a synthetic or biochemically derived nucleotide analog that is intrinsically fluorescent, e.g., as described in U.S. Pat. Nos. 6,268,132 and 5,763,167, Hawkins et al. (1995, Nucleic Acids Research, 23: 2872-2880), Seela et al. (2000, Helvetica Chimica Acta, 83: 910-927), Wierzchowski et al. (1996, Biochimica et Biophysica Acta, 1290: 9-17), Virta et al. (2003, Nucleosides, Nucleotides & Nucleic Acids, 22: 85-98), the entirety of each is hereby incorporated by reference. By “intrinsically fluorescent”, it is meant that the nucleotide analog is spectrally unique and distinct from the commonly occurring conventional nucleosides in their capacities for selective excitation and emission under physiological conditions. For the intrinsically fluorescent nucleotides, the fluorescence typically occurs at wavelengths in the near ultraviolet through the visible wavelengths. Preferably, fluorescence will occur at wavelengths between 250 nm and 700 nm and most preferably in the visible wavelengths between 250 nm and 500 nm.

The terms “detectable label” or “label” include a molecule or moiety capable of generating a detectable signal, either by itself or through the interaction with another label. The “label” may be a member of a signal generating system, and thus can generate a detectable signal in context with other members of the signal generating system, e.g., a biotin-avidin signal generation system, or a donor-acceptor pair for fluorescent resonance energy transfer (FRET) (Stryer et al., 1978, Ann. Rev. Biochem., 47:819; Selvin, 1995, Methods Enzymol., 246:300).

The term “nucleotide,” as used herein, refers to a phosphate ester of a nucleoside, e.g., mono, di, tri, and tetraphosphate esters, wherein the most common site of esterification is the hydroxyl group attached to the C-5 position of the pentose (or equivalent position of a non-pentose “sugar moiety”). The term “nucleotide” includes both a conventional nucleotide and a non-conventional nucleotide which includes, but is not limited to, phosphorothioate, phosphite, ring atom modified derivatives, and the like, e.g., an intrinsically fluorescent nucleotide.

As used herein, the term “conventional nucleotide” refers to one of the “naturally occurring” deoxynucleotides (dNTPs), including dATP, dTTP, dCTP, dGTP, dUTP, and dITP.

As used herein, the term “nonextendable nucleotide” refers to nucleotides which prevent extention of a polynucleotide chain by a polymerase. Examples of such nucleotides include dideoxy nucleotides (ddA, ddT, ddG, ddC) that lack a 3′-hydroxyl on the ribose ring, thereby preventing 3′ extension by DNA polymerases. Other examples of such nucleotides include but are not limited to inverted bases, which can be incorporated at the 3′-end of an oligo, leading to a 3′-3′ linkage which inhibits extension by DNA polymerases.

As used herein, the term “non-conventional nucleotide” refers to a nucleotide which is not a naturally occurring nucleotide. The term “naturally occurring” refers to a nucleotide that exists in nature without human intervention. In contradistinction, the term “non-conventional nucleotide” refers to a nucleotide that exists only with human intervention. A “non-conventional nucleotide” may include a nucleotide in which the pentose sugar and/or one or more of the phosphate esters is replaced with a respective analog. Exemplary pentose sugar analogs are those previously described in conjunction with nucleoside analogs. Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. A non-conventional nucleotide may show a preference of base pairing with another artificial nucleotide over a conventional nucleotide (e.g., as described in Ohtsuki et al. 2001, Proc. Natl. Acad. Sci., 98: 4922-4925, hereby incorporated by reference). The base pairing ability may be measured by the T7 transcription assay as described in Ohtsuki et al. (supra). Other non-limiting examples of “artificial nucleotides” may be found in Lutz et al. (1998) Bioorg. Med. Chem. Lett., 8: 1149-1152); Voegel and Benner (1996) Helv. Chim. Acta 76, 1863-1880; Horlacher et al. (1995) Proc. Natl. Acad. Sci., 92: 6329-6333; Switzer et al. (1993), Biochemistry 32:10489-10496; Tor and Dervan (1993) J. Am. Chem. Soc. 115: 4461-4467; Piccirilli et al. (1991) Biochemistry 30: 10350-10356; Switzer et al. (1989) J. Am. Chem. Soc. 111: 8322-8323, all of which hereby incorporated by reference. A “non-conventional nucleotide” may also be a degenerate nucleotide or an intrinsically fluorescent nucleotide.

As used herein, “isolated” or “purified” when used in reference to a polynucleotide means that a naturally occurring sequence has been removed from its normal cellular environment or is synthesized in a non-natural environment (e.g., artificially synthesized). Thus, an “isolated” or “purified” sequence may be in a cell-free solution or placed in a different cellular environment. The term “purified” does not imply that the sequence is the only polynucleotide present, but that it is essentially free (about 90-95%, up to 99-100% pure) of non-nucleotide or polynucleotide material naturally associated with it.

As used herein, “complementary” refers to the ability of a single strand of a polynucleotide (or portion thereof) to hybridize to an anti-parallel polynucleotide strand (or portion thereof) by contiguous base-pairing between the nucleotides (that is not interrupted by any unpaired nucleotides) of the anti-parallel polynucleotide single strands, thereby forming a double-stranded polynucleotide between the complementary strands. A first polynucleotide is said to be “completely complementary” to a second polynucleotide strand if each and every nucleotide of the first polynucleotide forms base-paring with nucleotides within the complementary region of the second polynucleotide. A first polynucleotide is not completely complementary (i.e., partially complementary) to the second polynucleotide if one nucleotide in the first polynucleotide does not base pair with the corresponding nucleotide in the second polynucleotide. The degree of complementarity between polynucleotide strands has significant effects on the efficiency and strength of annealing or hybridization between polynucleotide strands. This is of particular importance in amplification reactions, which depend upon binding between polynucleotide strands.

An oligonucleotide primer is “complementary” to a target polynucleotide if at least 50% (preferably, 60%, more preferably 70%, 80%, still more preferably 90% or more, up to and including to 100%) of the nucleotides of the primer form base-pairs with nucleotides on the target polynucleotide. Alternatively, or in addition, a sequence, e.g., a sequence in the 3′ end of an oligonucleotide template as described herein, is “complementary” to a target RNA sequence if it can hybridize selectively to the target RNA and permit polymerase extension of the hybridized target RNA under conditions suitable for a given polymerase.

As used herein, the term “analyzing,” when used in the context of a transcription reaction, refers to a qualitative (i.e., presence or absence, size detection, or identity etc.) or quantitative (i.e., amount) determination of a target polynucleotide, which may be visual or automated assessments based upon the magnitude (strength) or number of signals generated by the label. The “amount” (e.g., measured in μg, μmol or copy number) of a polynucleotide may be measured by methods well known in the art (e.g., by UV absorption, by comparing band intensity on a gel with a reference of known length and amount), for example, as described in Basic Methods in Molecular Biology, (1986, Davis et al., Elsevier, N.Y.); and Current Protocols in Molecular Biology (1997, Ausubel et al., John Weley & Sons, Inc.). One way of measuring the amount of a polynucleotide in the methods described herein is to measure the fluorescence intensity emitted by such polynucleotide, and compare it with the fluorescence intensity emitted by a reference polynucleotide, i.e., a polynucleotide with a known amount.

The practice of the methods described herein will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Polynucleotide Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995). The practice of the methods described herein may also involve techniques and compositions as disclosed in U.S. Pat. Nos. 5,965,409; 5,665,547; 5,262,311; 5,599,672; 5,580,726; 6,045,998; 5,994,076; 5,962,211; 6,217,731; 6,001,230; 5,963,456; 5,246,577; 5,126,025; 5,364,521; 4,985,129; as well as in U.S. patent applications 10/113,034; 10/387,286; 10/719,185; 10/600,201; 10/752,123 and 10/719,746. All patents, patent applications, and publications mentioned herein, both supra and infra, are hereby incorporated by reference.

### BRIEF DESCRIPTION OF THE FIGURES:

FIG. 1 shows a schematic diagram of one embodiment of the methods described herein.

FIG. 2 shows a schematic diagram of an embodiment of the methods described herein in which the oligonucleotide template is an RNA molecule.

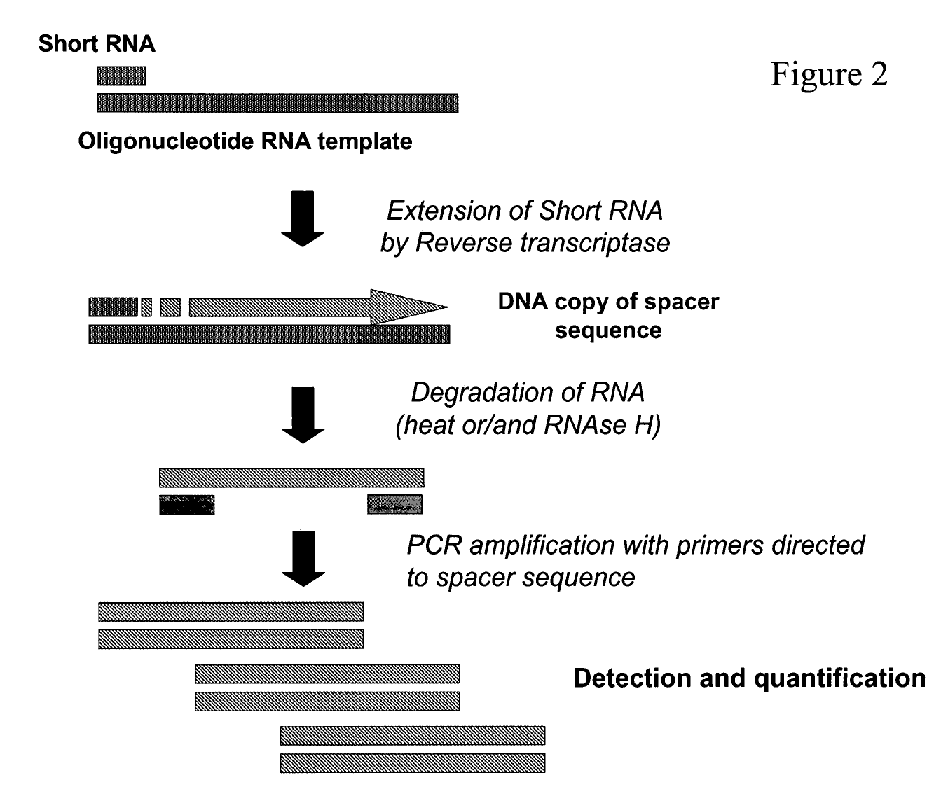

FIG. 3 shows a schematic diagram of an embodiment of the methods described herein in which the oligonucleotide template is a DNA molecule.

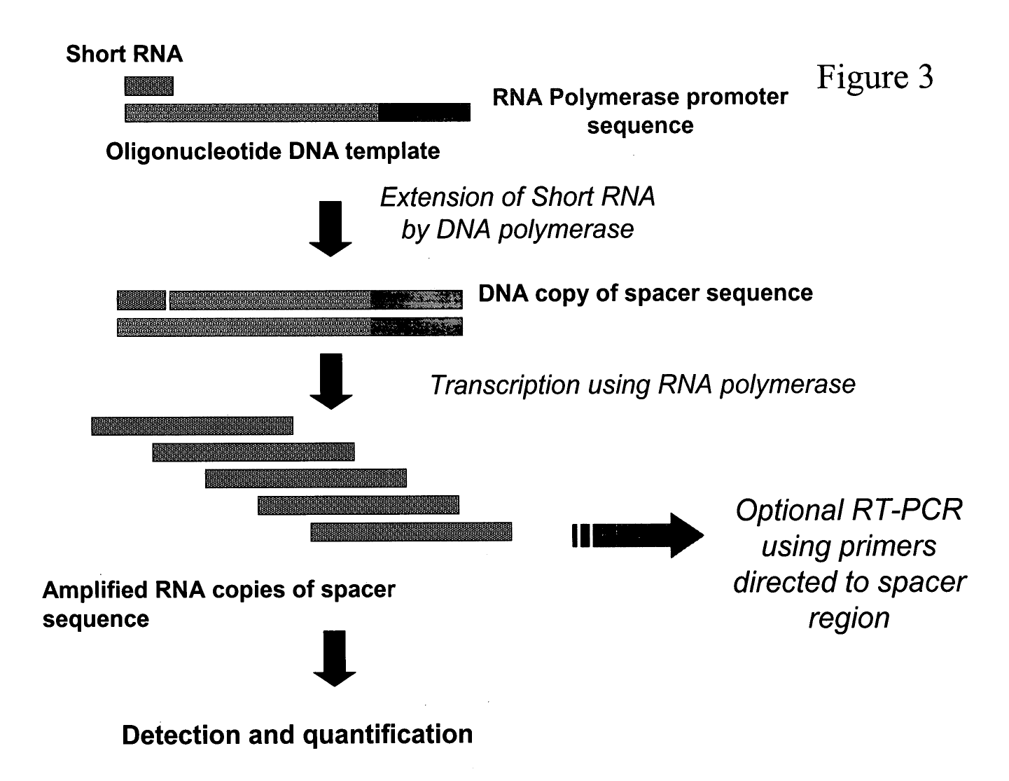

### DETAILED DESCRIPTION OF THE INVENTION

Described herein are approaches to the detection and quantitation of short RNAs in a biological sample. The methods permit the detection and quantitation of individual species of short RNA in a nucleic acid sample, both singly and in a multiplex format that permits the determination of expression levels for two or more target short RNAs in a single reaction.

The detection of multiple individual species of short RNAs in a nucleic acid sample may be accomplished by size separation of the amplified products of short RNA extension by, for example, capillary electrophoresis, coupled with detection by, for example, fluorescence detection. Quantitation of detected short RNA species can be accomplished by generating a standard curve by applying the methods described herein to samples containing known short RNA species in various concentrations. The standard curve permits the determination of the short RNA concentration(s) in the original sample.

The methods described herein provide for methods of identifying and/or detecting, and/or quantitating a suspected short RNA which has a 5′ and a 3′ end in a sample of interest comprising the following steps which are preferably performed contemporaneously, but alternatively may be separated in time. e.g., as when probe hybridization and extension of the short RNA are performed first, followed some time later (e.g., hours, days, etc.) by amplification and detection. The first step involves hybridizing an oligonucleotide template to the sample under conditions which permit the oligonucleotide template to bind to the short RNA. In this embodiment, the oligonucleotide template is an RNA molecule which has a 3′ and a 5′ end, and which comprises in order from its 3′ end to its 5′ end, a sequence complementary to the short RNA, and a spacer sequence adjacent to the complementary sequence.

Subsequently, the short RNA target is extended using a reverse transcriptase or DNA polymerase with reverse transcriptase activity to produce an extended short RNA product. The extended short RNA product comprises the short RNA and an extension adjacent to the 3′ end of the miRNA. The extension comprises, in order from its 5′ end to its 3′ end, a DNA sequence complementary to the spacer. The extension product can be detected and quantified using DNA amplification methods known in the art such as Polymerase chain reaction (PCR), Strand-displacement amplification (SDA) or Rolling circle amplification (RCA). Preferably, a detection method is polymerase chain reaction. More preferably, a detection and quantitation is real-time PCR as taught, for example in U.S. Pat. Nos. 5210015, 5487972, 5804375 , 5994056, 5538848 and 6030787.

Two or more short RNA sequences can be detected in a single reaction by using two specific RNA template sequences which contain different sequences in the spacer region which will be targeted by PCR primer and/or probes specific to one or another of the target RNA templates. In other embodiments, the template can be a DNA molecule. In another embodiment, multiplexing can be achieved by using the same sequences in the spacer region of specific RNA templates, where these sequences are separated by linkers of different known length, thereby correlating the length of the extension/amplification products with specific RNA targets. Thus, in this case PCR amplification can be conducted using the same PCR primers producing PCR products of different size which will be specific for or correlate with individual targeted short RNAs. The amplified PCR products can be separated by methods providing size discrimination such as electrophoresis or chromatography. Quantitation of amplified PCR products can be used to determine the initial amount of short RNAs by constructing a calibration curve by plotting known initial amount of short RNA versus the quantitity of amplified PCR product. Alternatively the initial amount of short RNA sequence can be determined by monitoring PCR amplification as described in U.S. Pat. No. 7081339.

In another embodiment the oligonucleotide template has a 3′ and a 5′ end, and comprises in order from its 3′ end to its 5′ end, a sequence complementary to the short RNA, a spacer of defined length and sequence, and a sequence encoding an RNA polymerase promoter.

Subsequent to annealing of target RNA to the oligonucleotide template, the short RNA is extended using a DNA polymerase to produce an extended short RNA product. The extended short RNA product comprises the short RNA and an extension adjacent to the 3′ end of the short RNA. The extension comprises, in order from its 5′ end to its 3′ end, a sequence complementary to the spacer of defined length and a sequence complimentary to an RNA polymerase promoter.

The extended short RNA product is transcribed using an RNA polymerase which recognizes the RNA polymerase promoter, thereby producing an RNA product. The resulting RNA product comprises in order from its 3′ to 5′ end, the complement of the spacer of defined length and sequence, and a sequence complementary to the complete short RNA. The detection of the RNA product is indicative of the presence of the suspected short RNA in the nucleic acid sample of interest and can be further used for quantitation of short RNA

The method described above can be adapted to provide analysis of two or more species (i.e., a plurality, e.g., 2, 3, 4, 5, 6, 7, 9, 9, 10, 15, 20, 25, 30, 40, 50, 75, 100 or more) of short RNAs from a single sample by varying the length of the spacer of defined length in each species of template, such that each template species has a unique length. When multiple species of oligonucleotides are used as templates for producing extended short RNA products in accordance with these methods, the transcript lengths generated by the transcription of these extended short RNA products differ. The separation of the transcribed products according to size allows the identification of the distinct transcripts which correlate with distinct species of short RNAs in the sample. In one aspect, the relative sizes of the short RNA products are distinguishable by electrophoresis or capillary electrophoresis.

### Hybridization

In a method comprising identifying a short RNA by means of analyzing an RNA product transcribed from the suspected short RNA molecule, an oligonucleotide template is hybridized to the suspected short RNA in the sample under hybridization conditions wherein the template is capable of binding specifically to the short RNA. Specifically, this hybridizing template has a 3′ and a 5′ end, and comprises in order from its 3′ end to its 5′ end, a sequence complementary to at least the 3′ portion of the short RNA of interest, a spacer of defined length and sequence, and, in one embodiment, a sequence encoding an RNA polymerase promoter, e.g., a prokaryotic RNA polymerase promoter, such as a bacterial, viral or bacteriophage RNA polymerase promoter. A spacer of defined length can be comprised of at least 1 nucleotide, or analogue thereof, and alternatively can comprise up to 2, 3, 4, 5, 6, 7, 8, 9, 10, 11, 12, 13, 14, 15, 16, 17, 18, 19, 20, 21, 22, 23, 24, 25, 30, 40, 50, 75, 100, 200, 300, 400, 500, 750, 1000, or up to approximately 1250 nucleotides or analogues thereof. The sequence encoding an RNA polymerase promoter can be for any polymerase capable of transcribing RNA, including, for example, T7 RNA polymerase, T3 RNA polymerase, SP6 RNA polymerase, and SP2 RNA polymerase.

Hybridization conditions useful in the methods described herein are well known to those of skill in the art and are described briefly below.

Hybridization can be performed at elevated temperatures (such as 40-70 degree C) to provide conditions under which only perfectly matched sequences of short RNAs and corresponding probes will form a double-stranded complex. Hybridization can be preceded by brief exposure to denaturing temperature conditions (such as heating to 80-90 degree C) to relax secondary structures in short RNA or to release short RNA from pre-existing complexes.

### Extension of Short RNA Sequences

Using the hybridized oligonucleotide described above as a template, the short RNA is extended from its 3′ end using a DNA polymerase or Reverse Transcriptase in the presence of nucleotides to produce a nucleic acid extension product. The extension product comprises the short RNA and an extension adjacent to the 3′ end of the miRNA. The extension comprises or consists of, in order from its 5′ end to its 3′ end, a sequence complementary to the spacer sequence, and, in one embodiment, a sequence comprising an RNA polymerase promoter. In the latter case, extension creates a double-stranded DNA molecule which includes a promoter for an RNA polymerase.

To prevent self-extension of the DNA oligonucleotide, which is used as a template sequence, the oligonucleotide can include a non-extendable base at its 3′-end such as dideoxy nucleotide or inverted base.

Extention can be performed at an elevated temperature to preserve specificity of hybridization, ensuring that only perfectly matched short RNA sequences will be extended by the DNA polymerase.

### Transcription of the Extended Short RNA product

In one embodiment, in order to detect a target short RNA, the extended short RNA product formed as described above is transcribed using an RNA polymerase which recognizes the RNA polymerase promoter located at the opposite end of the extension product, such that an RNA product is formed comprising in order from its 5′ to 3′ end, the spacer of defined length and a sequence complementary to the target short RNA. Preferably the transcription reaction occurs in the presence of ribonucleotides, including labeled ribonucleotides. In one aspect, the nucleotides are labeled.

The technique of in vitro transcription is well known to those of skill in the art. Briefly, the sequence of interest, in this instance, the short RNA DNA extension product comprising spacer of defined length, is linked to a promoter sequence for a prokaryotic polymerase, such as the bacteriophage T7, T3 and Sp6 RNA polymerase promoter, followed by in vitro transcription of the DNA template using the appropriate polymerase. The in vitro transcription reaction is performed, e.g., by incubating the linear DNA with transcription buffer (200 mM Tris-HCl, pH 8.0, 40 mM MgCl₂, 10 mM spermidine, 250 NaCl [T7 or T3] or 200 mM Tris-HCl, pH 7.5, 30 mM MgCl₂, 10 mM spermidine [Sp6]), dithiothreitol, RNase inhibitors, each of the four ribonucleoside triphosphates, and an RNA polymerase such as Sp6, T7 or T3 for 30 min at 37° C. In the method described herein, the transcription product is detected by evidence of the presence of a target short RNA. Quantitation of the transcript by reference to a control transcribed under similar conditions can provide an estimate of the abundance of the short RNA in the original sample.

If it is desired to prepare a labeled polynucleotide comprising RNA, unlabeled UTP can be omitted and replaced with or mixed with labeled UTP . Labels can include, for example, fluorescent labels or radiolabels.

Where isolation of the transcribed RNA is desired, the DNA template can be removed by incubation with a DNase. Phenol extraction can be used to remove the DNAse and polymerase, followed by precipitation and quantitation of the RNA, e.g., by UV absorption and/or by electrophoresis and visualization relative to known standards

Detection of the Transcribed Product

The detection of the transcribed product described above can be accomplished by any means known to one of skill in the art. Preferably, the detection is accomplished using detection of a label incorporated into the transcript. The detection of the labeled transcript indicates the presence of the short RNA of interest in the sample. Preferably, the detection is performed after or concurrently with size separation of the transcription products.

Size separation of nucleic acids is well known, e.g., by agars or polyacrylamide electrophoresis or by column chromatography, including HPLC separation. A preferred approach uses capillary electrophoresis, which is both rapid and accurate, readily achieving separation of molecules differing in size by as little as one nucleotide. Capillary electrophoresis uses small amounts of sample and is well-adapted for detection by, for example, fluorescence detection. Capillary electrophoresis is well known in the art and is described in further detail herein below.

As discussed above, transcribed nucleic acids corresponding to the target short RNA are preferably detected after separation. The detection notes both the position of a given band of nucleic acid of a given size and the abundance of that nucleic acid by, for example, UV absorption or, preferably, fluorescent signal. Fluorescent nucleotides can be incorporated into the amplified nucleic acid by simply adding one or more such nucleotides to the transcription reaction mixture prior to or during transcription.

The methods described herein are particularly adapted to detecting and/or quantifying multiple species of short RNAs in an individual sample. Each template is designed to detect a specific short RNA, and comprises sequences complementary to the short RNA of interest and a spacer of defined length particular to that template. That is, the length of the spacer of defined length in a primer which hybridizes to one species of short RNA, is designed so that it differs from the length of the spacer of defined length in a primer which hybridizes to a second, different species of short RNA. The extension products formed using these templates will differ, as will the length of the RNA transcript produced from the extension product. Thus, the two or more transcription products can be detected and quantitated in a single analysis when the transcripts are size separated and detected, e.g., by incorporation of a label.

For maximal sensitivity of the assay the described detection method can be combined with other amplification methods known in the art to amplify transcribed cRNA products. These products can be further amplified using Transcription-Mediated Amplification (TMA). Alternatively, the reaction mixture can be treated with heat-sensitive DNA nuclease (such as DNAse I) to destroy DNA template and then subjected to RT-PCR with primers directed to the spacer sequence. Quantitation can be performed by measuring the amount of amplified PCR product or by monitoring amplification process using Real-Time PCR methods.

Polymerases:

A wide variety of DNA polymerases can be used in the methods described herein. Suitable DNA polymerases for use in the subject methods may or may not be thermostable. Suitable polymerases will often be one of many polymerases commonly used in the field, and commercially available, such as DNA pol 1, Klenow fragment, T7 DNA polymerase, T4 DNA polymerase and Bst DNA polymerase. In addition, thermostable DNA polymerases, such as Taq, Vent, Pfu polymerase or other DNA polymerase derived from thermophylic microorganisms can be used. Preferably, the DNA polymerase lacks 5′-nuclease activity known to degrade RNA primers. Examples of such polymerases include Klenow fragment of DNA polymerase 1, Stoeffel fragment of Taq polymerase, Pfu polymerase or Vent polymerase. In some embodiments, a thermoactivated DNA polymerase typically referred to as “hot-start” DNA polymerase can be used to perform extention at elevated temperature.

A number of reverse trascriptases (RTases) can be used in the methods described herein. Suitable RTases are commercially available and can be employed to conduct the primer extension at a range of temperatures. The examples of available RTase include but are not limited to AMV, MMLV and HIV reverse transcriptases, thermostable RTases such as ThermoScript™ RNase H-Reverse Transcriptase and Thermo-X™ Reverse Transcriptase (Invitrogen), Transcriptor Reverse Transcriptase (Roche Applied Sciences). In addition, several thermostable DNA polymerases which display high reverse transcriptase activity such as Tht DNA Polymerase can be used to conduct reverse transcription and PCR amplification using a single enzyme.

For transcription, a number of RNA polymerases are also commercially available, such as T7 RNA polymerase, T3 polymerase, and SP6 RNA polymerase. Guidance for the use of such polymerases can readily be found in product literature and in general molecular biology guides such as Sambrook or Ausubel, both supra. Polymerases can incorporate labeled (e.g., fluorescent) nucleotides or their analogs during synthesis of polynucleotides, see, e.g., Hawkins et al., U.S. Pat. No. 5,525,711.

Template Design:

The methods described herein rely upon the use of DNA oligonucleotide templates for the extension of miRNA. Oligonucleotide templates for use in these methods can be designed according to general guidance well known in the art, as well as with specific requirements as described herein.

1. General Strategies for Template Design

Numerous factors influence the efficiency and selectivity of hybridization of a template to a second polynucleotide (target) molecule. These factors, which include template and target length, nucleotide sequence and/or composition, hybridization temperature, buffer composition and potential for steric hindrance in the region to which the template is required to hybridize, are considered when designing oligonucleotide templates useful in the methods described herein.

In one aspect, templates suitable for extending short RNAs include the following sequence from 3′ to 5′: a) sequences complementary to the short RNA target, including the 3′ end of the short RNA, a spacer of defined length ranging from 1 to about 1200 nucleotides, and an RNA polymerase recognition site. Templates useful in the methods described herein have a particular melting temperature (Tₘ) which can be useful in predicting or maximizing specificity. Tₘ can be estimated using, e.g., commercial programs, including, e.g., Oligo-dT Obliged, Primer Design and programs available on the internet, including Primer 3 and Oligo Calculator. Preferably, the Tm of a template useful in the methods described herein, or more particularly the Tm of the 3′ stretch complementary to the target short RNA, is between about 45 and 65° C. and more preferably between about 50 and 60° C.

Tₘ of a polynucleotide affects its hybridization to another polynucleotide (e.g., the annealing of a target short RNA to a template polynucleotide). In the subject methods, it is necessary that the oligonucleotide template used selectively hybridizes to a target short RNA. Typically, selective hybridization occurs when two polynucleotide sequences are substantially complementary (at least 65% complementary over a stretch of at least 14 to 25 nucleotides, preferably at least 75%, more preferably at least 90%, and more preferably still, 100% complementary). See Kanehisa, M., 1984, Polynucleotides Res. 12: 203, incorporated herein by reference. Preferably, the portion of the template complementary to the target short RNA is complementary to the full length (i.e., 21, 22 bases) of the short RNA, e.g., an miRNA. It is important that at least the 3′ nucleotide of the short RNA hybridizes to the template in order to permit primer extension. Longer stretches of complementarity provide greater specificity of hybridization as a general rule. So it is preferred in order to maximize specificity, that the template be complementary to more, rather than less of the short RNA, e.g., at least 15 nucleotides, preferably at least 18 nucleotides, 19 nucleotides, 20 nucleotides, 21 nucleotides, or 22 nucleotides or more (including, for example, the full length of a common miRNA or other short RNA target).

As noted above, a positive correlation exists between complementary template length and both the efficiency and accuracy with which a template will anneal to a target sequence. In particular, longer sequences have a higher melting temperature (Tₘ) than do shorter ones, and are less likely to be repeated within a given target sequence, thereby minimizing promiscuous hybridization. Thus it is preferred that the full complementary sequence of a target short RNA be represented in the 3′ region of a template useful in the method described herein. It is emphasized that the critical region of the template, at least with regard to specificity for a target short RNA, is the region complementary to the short RNA. There is thus a limited amount of manipulation possible for the short RNA-complementary region of the template, but it can be helpful to consider the full length of the template to avoid problems such as internal self-complementarity, etc. Template sequences with a high G-C content or that comprise palindromic sequences tend to self-hybridize, as do their intended target sites, since unimolecular, rather than bimolecular, hybridization kinetics are generally favored in solution. However, it is also important to design a template that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with annealing efficiency, as does the concentration of organic solvents, e.g. formamide, that might be included in a priming reaction or hybridization mixture, while increases in salt concentration facilitate binding. Under stringent annealing conditions, longer hybridization probes, or synthesis primers, hybridize more efficiently than do shorter ones, which are sufficient under more permissive conditions. Preferably, stringent hybridization is performed in a suitable buffer under conditions that allow the short RNA to hybridize to the oligonucleotide template. Stringent hybridization conditions can vary (for example from salt concentrations of less than about 1M, more usually less than about 500 mM and preferably less than about 200 mM) and hybridization temperatures can range (for example, from as low as 0° C. to greater than 22° C., greater than about 30° C., and (most often) in excess of about 37° C.) depending upon the lengths and/or the polynucleotide composition or the hybridizing sequences. Longer target RNAs may require higher hybridization temperatures for specific hybridization. As several factors affect the stringency of hybridization, the combination of parameters is more important than the absolute measure of a single factor.

The design of a template, or a set of templates, useful in the methods described herein can be facilitated by the use of readily available computer programs, developed to assist in the evaluation of the several parameters described above and the optimization of primer sequences. Examples of such programs are “PrimerSelect” of the DNAStar™ software package (DNAStar, Inc.; Madison, Wis.), OLIGO 4.0 (National Biosciences, Inc.), PRIMER, Oligonucleotide Selection Program, PGEN and Amplify (described in Ausubel et al., supra).

2. Oligonucleotide Synthesis

The oligonucleotide templates and amplification primers themselves are synthesized using techniques that are also well known in the art. Methods for preparing oligonucleotides of specific sequence include, for example, cloning and restriction digestion of appropriate sequences and direct chemical synthesis. Once designed, oligonucleotides can also be prepared by a suitable chemical synthesis method, including, for example, the phosphotriester method described by Narang et al., 1979, Methods in Enzymology, 68 : 90, the phosphodiester method disclosed by Brown et al., 1979, Methods in Enzymology, 68: 109, the diethylphosphoramidate method disclosed in Beaucage et al., 1981, Tetrahedron Letters, 22: 1859, and the solid support method disclosed in U.S. Pat. No. 4,458,066, or by other chemical methods using either a commercial automated oligonucleotide synthesizer (which is commercially available) or VLSIPS™ technology.

Sample Preparation

The sample for use in the above methods contains short RNAs. In most instances, there will need to be a step of isolating nucleic acid from a given sample source, to provide nucleic acid including target short RNAs, in a form accessible to hybridization with a primer. Typically, these methods of isolation involve cell lysis, followed by purification of polynucleotides by methods such as phenol/chloroform extraction, electrophoresis, and/or chromatography. Often, such methods include a step wherein the polynucleotides are precipitated, e.g. with ethanol, and resuspended in an appropriate buffer for primer extension, or similar reaction.

In certain embodiments, two or more target polynucleotides from one or more sample sources are analyzed in a single reaction. In some applications, a single polynucleotide from a multitude of sources may be synthesized to screen for the presence or absence of a particular sequence. In other applications, a plurality of polynucleotides may be generated from a single sample or individual, thereby allowing the assessment of a variety of polynucleotides in a single sample, e.g., to simultaneously screen for a multitude of disease markers in an individual. Any of the above applications can be easily accomplished using the methods described herein.

Thus, a reaction mixture may comprise one target polynucleotide, or it may comprise two or more different target polynucleotides. The present method allows for simultaneous analysis of two or more polynucleotides obtained from a plurality of samples, i.e., multiplex analysis.

Once the starting cells, tissues, organs or other samples are obtained, RNA or cDNAs can be prepared there from by methods that are well-known in the art.

Where desired or necessary, RNA can be purified, for example from tissues, according to the following guanidinium isothiocyanate method. Following removal of the tissue of interest, pieces of tissue of ≦2 g are cut and quick frozen in liquid nitrogen, to prevent degradation of RNA. Upon the addition of a suitable volume of guanidinium solution (for example 20 ml guanidinium solution per 2 g of tissue), tissue samples are ground in a tissuemizer with two or three 10-second bursts. To prepare tissue guanidinium solution (1 L) 590.8 g guanidinium isothiocyanate is dissolved in approximately 400 ml DEPC-treated H₂O. 25 ml of 2 M Tris-HCl, pH 7.5 (0.05 M final) and 20 ml Na₂EDTA (0.01 M final) is added, the solution is stirred overnight, the volume is adjusted to 950 ml, and 50 ml 2-ME is added.

Homogenized tissue samples are subjected to centrifugation for 10 min at 12,000×g at 12° C. The resulting supernatant is incubated for 2 min at 65° C. in the presence of 0.1 volume of 20% Sarkosyl, layered over 9 ml of a 5.7M CsCl solution (0.1 g CsCl/ml), and separated by centrifugation overnight at 113,000×g at 22° C. After careful removal of the supernatant, the tube is inverted and drained. The bottom of the tube (containing the RNA pellet) is placed in a 50 ml plastic tube and incubated overnight (or longer) at 4° C. in the presence of 3 ml tissue resuspension buffer (5 mM EDTA, 0.5% (v/v) Sarkosyl, 5% (v/v) 2-ME) to allow complete resuspension of the RNA pellet. The resulting RNA solution is extracted sequentially with 25:24:1 phenol/chloroform/isoamyl alcohol, followed by 24:1 chloroform/isoaamyl alcohol, precipitated by the addition of 3 M sodium acetate, pH 5.2, and 2.5 volumes of 100% ethanol, and resuspended in DEPC water (Chirgwin et al., 1979, Biochemistry, 18: 5294).

Alternatively, RNA is isolated from tissues according to the following single step protocol. The tissue of interest is prepared by homogenization in a glass teflon homogenizer in 1 ml denaturing solution (4M guanidinium thiosulfate, 25 mM sodium citrate, pH 7.0, 0.1 M 2-ME, 0.5% (w/v) N-laurylsarkosine) per 100 mg tissue. Following transfer of the homogenate to a 5-ml polypropylene tube, 0.1 ml of 2 M sodium acetate, pH 4, 1 ml water-saturated phenol, and 0.2 ml of 49:1 chloroform/isoamyl alcohol are added sequentially. The sample is mixed after the addition of each component, and incubated for 15 min at 0-4° C. after all components have been added. The sample is separated by centrifugation for 20 min at 10,000×g, 4° C., precipitated by the addition of 1 ml of 100% isopropanol, incubated for 30 minutes at −20° C. and pelleted by centrifugation for 10 minutes at 10,000×g, 4° C. The resulting RNA pellet is dissolved in 0.3 ml denaturing solution, transferred to a microfuge tube, precipitated by the addition of 0.3 ml of 100% isopropanol for 30 minutes at −20° C., and centrifuged for 10 minutes at 10,000×g at 4° C. The RNA pellet is washed in 70% ethanol, dried, and resuspended in 100-200 μl DEPC-treated water or DEPC-treated 0.5% SDS (Chomczynski and Sacchi, 1987, Anal. Biochem., 162: 156).

Kits and reagents for isolating total RNAs are commercially available from various companies, for example, RNA isolation kit (Stratagene, La Lola, Calif., Cat # 200345); PicoPure™ RNA Isolation Kit (Arcturus, Mountain View, Calif., Cat # KIT0202); and RNeasy Protect Mini, Midi, and Maxi Kits (Qiagen, Cat # 74124).

Kits and reagents for isolating miRNAs are commercially available from various companies, for example, the mirVana™ miRNA Isolation Kit (Ambion, Austin Tex., Cat. #1560) and PureLink™ miRNA Isolation Kit (Invitrogen, Carlsbad, Calif., Cat. #K157001)

In some embodiments, total RNAs are used in the subject method. In other embodiments, the sample can be fractionated to remove or enrich for one or more components, e.g., miRNA, rRNA, etc. Kits and reagents for measuring or isolating mRNAs are commercially available from, e.g., Oligotex MRNA Kits (Qiagen, Cat # 70022).

Labeled Nucleotides

The method described herein can benefit from the incorporation of one or more labeled nucleotides. The label preferably includes a fluorescent label. A labeled nucleotide can be a fluorescent dye-linked nucleotide, or it can be an intrinsically fluorescent nucleotide. In one embodiment of the methods described herein, a conventional deoxynucleotide linked to a fluorescent dye is used. Non-limiting examples of some useful labeled nucleotides are listed in Table 1.

TABLE 1
Examples of labeled nucleotides

| Fluorescein Labeled | Fluorophore Labeled |
|---|---|
| Fluorescein - 12 - CTP | Eosin - 6 - CTP |
| Fluorescein - 12 - UTP | Coumarin - 5 -ddUTP |
| Fluorescein - 12 - ATP | Tetramethylrhodamine - UTP |
| Fluorescein - 12 - GTP | Texas Red - 5 - ATP |
| Fluorescein - N6 - ATP | LISSAMINETM - rhodamine - 5 - GTP |
| FAM Labeled | TAMRA Labeled |
| FAM - UTP | TAMRA - UTP |
| FAM - CTP | TAMRA - CTP |
| FAM - ATP | TAMRA - ATP |
| FAM - GTP | TAMRA - GTP |
| ROX Labeled | JOE Labeled |
| ROX - UTP | JOE - UTP |
| ROX - CTP | JOE - CTP |
| ROX - ATP | JOE - ATP |
| ROX - GTP | JOE - GTP |
| R6G Labeled | R110 Labeled |
| R6G - UTP | R110 - UTP |
| R6G - CTP | R110 - CTP |
| R6G - ATP | R110 - ATP |
| R6G - GTP | R110 - GTP |
| BIOTIN Labeled | DNP Labeled |
| Biotin - N6 - ATP | DNP - N6 - ATP |

Fluorescent dye-labeled nucleotides can be purchased from commercial sources. Labeled polynucleotides and nucleotide can also be prepared by any of a number of approaches known in the art.

Fluorescent dyes useful as detectable labels are well known to those skilled in the art and numerous examples can be found in the Handbook of Fluorescent Probes and Research Chemicals 6th Edition, Richard Haugland, Molecular Probes, Inc., 1996 (ISBN 0-9652240-0-7).

Preferably, fluorescent dyes are selected for compatibility with detection on an automated capillary electrophoresis apparatus and thus should be spectrally resolvable and not significantly interfere with electrophoretic analysis. Examples of suitable fluorescent dyes for use as detectable labels can be found, in among other places, U.S. Pat. Nos. 5,750,409; 5,366,860; 5,231,191; 5,840,999; 5,847,162; 4,439,356; 4,481,136; 5,188,934; 5,654,442; 5,840,999; 5,750,409; 5,066,580; 5,750,409; 5,366,860; 5,231,191; 5,840,999; 5,847,162; 5,486,616; 5,569,587; 5,569,766; 5,627;027; 5,321,130; 5,410,030; 5,436,134; 5,534,416; 5,582,977; 5,658,751; 5,656,449; 5,863,753; PCT Publications WO 97/36960; 99/27020; 99/16832; European Patent EP 0 050 684; Sauer et al, 1995, J. Fluorescence 5 : 247-261; Lee et al., 1992, Nucl. Acids Res. 20: 2471-2483; and Tu et al., 1998, Nucl. Acids Res. 26 : 2797-2802, all of which are incorporated herein in their entireties.

Nucleotide can be modified to include functional groups, such as primary and secondary amines, hydroxyl, nitro and carbonyl groups, for fluorescent dye linkage (see Table 2).

TABLE 2

| Functional Group | Reaction | Product |
|---|---|---|
| Amine | dye - isothiocyanates | Thiourea |
| Amine | dye - succinimidyl ester | Carboxamide |
| Amine | dye - sulfonyl chloride | Sulphonamide |
| Amine | dye - aldehyde | Alkylamine |
| Ketone | dye - hydrazides | Hydrazones |
| Ketone | dye - semicarbazides | Hydrazones |
| Ketone | dye - carbohydrazides | Hydrazones |
| Ketone | dye - amines | Alkylamine |
| Aldehyde | dye - hydrazides | Hydrazones |
| Aldehyde | dye - semicarbazides | Hydrazones |
| Aldehyde | dye - carbohydrazides | Hydrazones |
| Aldehyde | dye - amines | Alkylamine |
| Dehydrobutyrine | dye - sulphydryl | Methyl lanthionine |
| Dehydroalanine | dye - sulphydryl | Lanthionine |

Useful fluorophores include, but are not limited to: Texas Red™ (TR), Lissamine™ rhodamine B, Oregon Green™ 488 (2′,7′ -difluorofluorescein), carboxyrhodol and carboxyrhodamine, Oregon Green™ 500, 6 - JOE (6 - carboxy - 4′,5′-dichloro -2′,7′-dimethyoxyfluorescein, eosin F3S (6-carobxymethylthio-2′,4′,5′,7′-tetrabromo-trifluorofluorescein), cascade blue™ (CB), aminomethylcoumarin (AMC), pyrenes, dansyl chloride (5-dimethylaminonaphthalene-1-sulfonyl chloride) and other napththalenes, PyMPO, ITC (1-(3-isothiocyanatophenyl)-4-(5-(4-methoxyphenyl)oxazol-2-yl)pyridinium bromide), coumarin, fluorescein, tetrachlorofluorescein, hexachlorofluorescein, Lucifer yellow, rhodamine, BODIPY, tetramethylrhodamine, Cy3, Cy5, Cy7, eosine, and ROX. Combination fluorophores such as fluorescein-rhodamine dimers, described, for example, by Lee et al. (1997), Polynucleotides Research 25:2816, are also suitable. Fluorophores may be chosen to absorb and emit in the visible spectrum or outside the visible spectrum, such as in the ultraviolet or infrared ranges. Suitable fluorescent dye labels are commercially available from Molecular Probes, Inc., Eugene, Oreg., US and Research Organics, Inc., Cleveland, Ohio, US, among other sources, and can be found in the Handbook of Fluoresdent Probes and Research Chemicals 6th Edition, Richard Haugland, Molecular Probes, Inc., 1996 (ISBN 0-9652240-0-7).

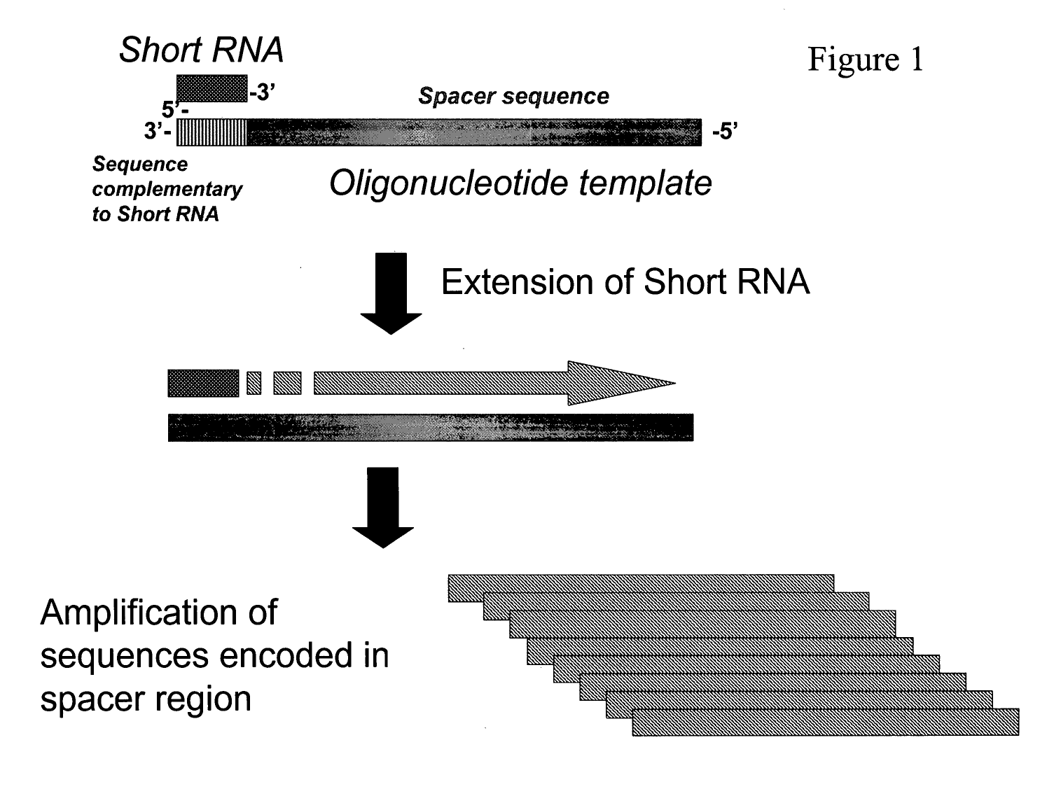

A labeled nucleotide useful in the methods described herein includes an intrinsically fluorescent nucleotide known in the art, e.g., the novel fluorescent nucleoside analogs as described in U.S. Pat. No. 6,268,132B1 (the entirety is hereby incorporated by reference). The fluorescent analogs of the U.S. Pat. No. 6,268,132B1 are of three general types: (A) C-nucleoside analogs; (B) N-nucleoside analogs; and (C) N-azanucleotide and N-deazanucleotide analogs. All of these compounds have three features in common: 1) they are structural analogs of the common nucleosides capable of replacing naturally occurring nucleosides in enzymatic or chemical synthesis of oligonucleotides; 2) they are naturally fluorescent when excited by light of the appropriate wavelength(s) and do not require additional chemical or enzymatic processes for their detection; and 3) they are spectrally distinct from the nucleosides commonly encountered in naturally occurring DNA. At least 125 specific compounds have been identified in U.S. Pat. No. 6,268,132B1. These compounds, which have been characterized according to their class, structure, chemical name, absorbance spectra, emission spectra, and method of synthesis, are tabulated as shown in FIGS. 21A-21F-1 of the U.S. Pat. No. 6,268,132B1.

The labeled nucleotide as described herein also includes, but is not limited to, fluorescent N-nucleosides and fluorescent structural analogs. Formycin A (generally referred to as Fonnycin), the prototypical fluorescent nucleoside analog, was originally isolated as an antitumor antibiotic from the culture filtrates of Nocardia interforma (Hori et al. [1966] J. Antibiotics, Ser. A 17:96-99) and its structure identified as 7-amino-3-b-D-ribafuranosyl (1H-pyrazolo-[4,3d] pyrimidine)) (FIGS. 5 and 6). This antibiotic, which has also been isolated from culture broths of Streptomyces lavendulae (Aizawa et al. [1965] Agr. Biol. Chem. 29:375-376), and Streptomyces gummaensis (Japanese Patent No. 10,928, issued in 1967 to Nippon Kayaku Co., Ltd.), is one of numerous microbial C-ribonucleoside analogs of the N-nucleosides commonly found in RNA from all sources. The other naturally-occurring C-ribonucleosides which have been isolated from microorganisms (FIG. 4) include formycin B (Koyama et al. [1966] Tetrahedron Lett. 597-602; Aizawa et al., supra; Umezawa et al. [1965] Antibiotics Ser. A 18:178-181), oxoformycin B (Ishizuka et al. [1968] J. Antibiotics 21:1-4; Sawa et al. [1968] Antibiotics 21:334-339), pseudouridine (Uematsu and Suahdolnik [1972] Biochemistry 11:4669-4674), showdomycin (Darnall et al. [1967] PNAS 57:548-553), pyrazomycin (Sweeny et al. [1973] Cancer Res. 33:2619-2623), and minimycin (Kusakabe et al. [1972] J. Antibiotics 25:44-47). Formycin, formycin B, and oxoformycin B are pyrazolopyrimidinenucleosides and are structural analogs of adenosine, inosine, and hypoxanthine, respectively; a pyrazopyrimidine structural analog of guanosine obtained from natural sources has not been reported in the literature. A thorough review of the biosynthesis of these compounds is available in Ochi et al. (1974) J. Antibiotics xxiv:909-916. The entirety of each reference is here by incorporated by reference.

Detection and quantitation of DNA products amplified by Polymerase Chain Reaction.

Methods for detecting and quantifying the amplified PCR products are well known in the art and any of them can be used in the methods described herein. The example of such methods and systems include real-time PCR with detection of amplified nucleic acid with fluorescent dyes binding to double stranded DNA, such as SYBR Green or ethidium bromide, Real-time PCR with molecular beacons (detecting binding of fluorescently labeled probes to adjacent sequence in amplified PCR products), Real-Time PCR using a 5′-nuclease assay with Taqman probes (Applied BioSystems, Foster City, Calif.), involving Real-Time PCR thermocyclers such as the Lightcycler system from Roche (Indianapolis, Ind.), Applied Biosystems 7900HT, 7300, 7500 Real-time PCR systems (Foster City, Calif.), 1-cycler from Bio-rad (, Rotorgene Real-time PCR cycler from Corbett(Sydney, Australia) and others.

Amplified PCR products can also be separated and quantified by electrophoresis and, preferably, by capillary electrophoresis as described below.

Separation and Detection of Transcribed Products:

Methods for detecting the presence or amount of polynucleotides are well known in the art and any of them can be used in the methods described herein so long as they are capable of separating individual polynucleotides by at least the difference in length as the size of a spacer of defined length which is used. It is preferred that the separation and detection permits detection of length differences as small as one nucleotide. It is further preferred that the separation and detection can be done in a high-throughput format that permits real time or contemporaneous determination of amplicon abundance in a plurality of reaction aliquots taken during the cycling reaction. Useful methods for the separation and analysis of the amplified products include, but are not limited to, electrophoresis (e.g., capillary electrophoresis (CE)), chromatography (dHPLC), and mass spectrometry.

In one embodiment, CE is a preferred separation means because it provides exceptional separation of the polynucleotides in the range of at least 10-1,000 base pairs with a resolution of up to a single base pair. CE can be performed by methods well known in the art, for example, as disclosed in U.S. Pat. Nos. 6,217,731; 6,001,230; and 5,963,456, which are incorporated herein by reference. High-throughput CE apparatuses are available commercially, for example, the HTS9610 High throughput analysis system and SCE 9610 fully automated 96-capillary electrophoresis genetic analysis system from Spectrumedix Corporation (State College, Pa.); P/ACE 5000 series and CEQ series from Beckman Instruments Inc (Fullerton, Calif.); and ABI PRISM 3100, 3130 and 3730 genetic analyzers (Applied Biosystems, Foster City, Calif.). Near the end of the CE column, in these devices the amplified DNA fragments pass a fluorescence detector which measures signals of fluorescent labels. These apparatuses provide automated high throughput for the detection of fluorescence-labeled PCR products.

The employment of CE in the methods described herein permits higher productivity compared to conventional slab gel electrophoresis. The separation speed is limited in slab gel electrophoresis because of the heat produced when the high electric field is applied to the gel. Since heat elimination is very rapid from the large surface area of a capillary, a higher electric field can be applied in capillary electrophoresis, thus accelerating the separation process. By using a capillary gel, the separation speed is increased about 10 fold over conventional slab-gel systems.

With CE, one can also analyze multiple samples at the same time, which is essential for high-throughput. This is achieved, for example, by employing multi-capillary systems.

In one embodiment, the methods described herein measure the relative amount of a particular short RNA contained in the sample. The detected signal strength following size separation can be recorded for each transcribed species in the reaction, using nucleic acid from two separate samples which will provide a relative number or measure of the abundance of the target short RNA in the samples.

The described methods of detecting a suspected short RNA in a sample can be useful in diagnostic or therapeutic applications. For example, short RNAs, such as miRNAs can be detected in biological samples of interest obtained from one or more eukaryotes, particularly in vertebrates and more particularly in mammals, including humans, experimental animals, e.g. mice, rats guinea pigs, suspected of having any one of a number of conditions diseases or disorders with which one or more micro RNAs which may be known or suspected to be associated. Further, samples may be identified as well from normal healthy control individuals not having said disease disorder or condition, and these samples may serve as controls for diseases, disorders, or conditions which are characterized by differential expression of miRNA-molecules. Samples may also be obtained from drosophila, fungi, etc.

The amount of a miRNA in a sample compared to a comparable control can be used in methods of identifying the miRNA as a biomarker of the disease, disorder or condition of interest. Alternatively the sample of interest can be from healthy individuals, and used to monitor different stages of development or aging. Further, the sample can be used to monitor the progression or staging of any number of diseases, conditions or developmental stages of interest.

The detection of specific miRNAs in samples (or, for that matter, the relative lack of such detection) can be used to identify the samples as containing cells or tissues of a certain class, or of a certain developmental stage, or of being infected, or of being in an a cancerous form. Samples encompassed by the methods include, but are not limited to, samples comprising, or derived from, cells and tissues of any vertebrate, including humans, including, for example, tissues and cells from kidney, liver, spleen, heart, skin, heart brain, neural tissues, and intestine, tissue sections, epithelia, endothelia, and the lymphatic system. Alternatively samples used in the methods disclosed may contain isolated or purified RNA from any of the aforementioned cells and tissues. Further still, the samples may contain miRNAs or other short RNAs that have been artificially synthesized.

### EXAMPLES

### Example 1

Extension using DNA templates:

Serial dilutions of let7-a miRNA (5′-UGAGGUAGUAGGUUGUAUAGUU-3′) are prepared to the concentration range 0.01 nM to 100 nM.

a) A 1 ul aliquot of let 7a miRNA solution is combined with a 1 ul aliquot of a 2 uM solution of DNA oligonucleotide Let7A-T72 (5′-CTAATACGACTCACTATAGGGAGAGCTGAAATCACAAATACAACGAATCGAG TAAACTATACAACCTACTACCTCA-3ddC-3″) in 20 ul of the reaction buffer: 10 mM Tris, 50 mM NaCl, 10 mM MgCl₂, 1 mM dithiothreitol, 0.2 mM dNTPs (desoxyribonucleotides triphosphates) (pH 7.9) containing 1 U of Klenow fragment of DNA Polymerase 1 (New England Biolabs, Beverly, Mass.) and incubated at 37° C. for 30 mm.

b) A 1 ul aliquot of the solution of let 7a miRNA is combined with a 1 ul aliquot of a 2 uM solution of DNA oligonucleotide Let7A-T72 (5′-CTAATACGACTCACTATAGGGAGAGCTGAAATCACAAATACAACGAATCGAG TAAACTATACAACCTACTACCTCA-3ddC-3″) in 20 ul of the reaction buffer: 20 mM Tris, 10 mM KCl, 10 mM (NH4)₂SO₄, 2 mM MgSO₄, 0.1% Triton X-100, 0.2 mM dNTPs (desoxyribonucleotides triphosphates) (pH 7.9) containing 10 U of Large fragment of Bst DNA Polymerase (New England Biolabs, Beverly, Mass.) and incubated at 60° C. for 30 mm.

c) A 1 ul aliquot of the solution of let 7a miRNA is combined with a 1 ul aliquot of a 2 uM solution of DNA oligonucleotide Let7A-T72 (5′-CTAATACGACTCACTATAGGGAGAGCTGAAATCACAAATACAACGAATCGAG TAAACTATACAACCTACTACCTCA-3ddC-3″) in 20 ul of the reaction buffer: 20 mM Tris, 10 mM KC1, 10 mM (NH₄)₂SO₄, 2 mM MgSO₄, 0.1% Triton X-100, 0.2 mM dNTPs (desoxyribonucleotides triphosphates) (pH 7.9) containing 1 U of Vent (exo-) DNA Polymerase (New England Biolabs, Beverly, Mass.). The reaction mixture is heated for 1 min at 90° C. and incubated for 30 min at 60° C. in a thermocycler.

Transcription:

Reaction mixtures are supplemented with 0.5 mM each ATP, CTP, GTP, 0.1 mM UTP and 0.01 mM Fluorescein-labeled UTP (Invitrogen), MgCl₂ to reach 10 mM and dithiothreitol to reach 10 mM. Finally, 50 U T7 RNA polymerase ((New England Biolabs, Beverly, Mass.) are added to the reaction. Reaction mixtures are further incubated at 37° C. for 3 hours.

Detection and Quantitation.

3 ul of reaction mixtures are mixed with 8 ul of formamide, supplemented with 0.3 ul of GeneScan 350 Rox size standards (Applied Biosystems, Foster City, Calif.) and injected on ABI 3130 Genetic Sequence Analyzer Capillary Electrophoresis system (Applied Biosystems, Foster City, Calif.). Electrophoresis on 36 cm capillaries is performed according to manufacture's instructions using POP4 or GeneScan polymer gels.

### Example 2

Multiplex detection of let 7a ( 5-UGAGGUAGUAGGWUGUAUAGUU) and let 7c miRNAs (5-UGAGGUAGUAGGUUGUAUGGUU) is performed as described above, with the following modifications: Combined serial dilutions of let 7a and let 7b are mixed with DNA templates Let7A-T72 (5′-CTAATACGACTCACTATAGGGAGAGCTGAAATCACAAATACAACGAATCGAG TAAACTATACAACCTACTACCTCA-3′ddC) and Let7C-T71 (5′-CTAATACGACTCACTATAGGGAGAGCGATAAATTAGAATTCGAACCATACAA CCTACTACCTCA-3′ddC).

### Example 3

Reverse transcription-PCR.

Different concentrations of serially diluted let 7a miRNA (1 pM-1 nM) are mixed with 2 ul of 1 nM RNA template: 5′-UAUUCCAGACUCACCUUAUACACAGCUGAAAUCACAAAUACAACGAAUCG AGUAAACUAUUCAACCUACUACCUCA in reaction buffer containing 50 mM Tris, 50 mM KCl, 2 mM MgCl2, 0.2 mM dNTPs and 2U of M-MLV Reverse Transcriptase (Promega, Madison, Wis.) and incubated for 20 min at 45-50° C. (total volume 50 ul). The reverse transcriptase is heat inactivated for 10 min at 80° C. and reaction is supplemented with 1 uM of oligonucleotide primers directed to the spacer region 5-TTACTCGATTGCTTGTATTTGT and 5-TTCCAGACTCACCTTATAC, 2 U Taq Polymerase (Promega, Madison, Wis.), and 5 ul of 1/10000 dilution of SYBR Green dye (Invitrogen).

The reaction is placed into an I-Cycler Real-Time PCR system (Biorad) and subjected to real-time PCR performed according to manufacturer's instruction. The amplification is conducted for 30 cycles comprising the following steps: 15 s at 95° C., 20 s at 50° C. and 60 sat 65° C.

1. A method of detecting a short RNA of interest in a nucleic acid sample, wherein said short RNA has a 5′ and a 3′ end, comprising:
a) hybridizing an oligonucleotide template to said sample under conditions wherein said template binds to said short RNA, said template having a 3′ and a 5′ end, said template comprising in order from its 3′ end to its 5′ end, a sequence complementary to said short RNA, and a spacer sequence;
b) extending said short RNA using a DNA polymerase to produce an extended short RNA product, such that said extended short RNA product comprises said short RNA and a sequence complementary to the spacer sequence;
c) subjecting said extended short RNA product to an amplification regimen, thereby producing a plurality of copies of a sequence present in said spacer sequence; and
d) detecting a spacer sequence amplification product produced in step (c), wherein the detection of an amplification product formed in step (c) indicates the presence of said short RNA sequence of interest in said nucleic acid.

2. A method of determining a quantity of a short RNA in a nucleic acid sample, the method comprising:
a) subjecting a plurality of different known quantities of said short RNA to the method of claim 1;
b) subjecting a sample for which the quantity of said short RNA is to be determined to the method of claim 1;
c) measuring the quantity of amplification products generated in step (a) for each of said known quantities of said short RNA;
d) comparing the quantity of amplification products produced in step (b) with the quantities of amplification products of step (c), wherein said comparing determines a quantity of said short RNA in said sample for which a quantity of said short RNA was to be determined.

3. The method of claim 1, wherein said short RNA is a microRNA (miRNA).

4. The method of claim 1, wherein said short RNA is a short interfering RNA (siRNA) or an RNA molecules derived from siRNA metabolic degradation.

5. The method of claim 1, wherein said short RNA is a short hairpin RNA (shRNA) or an RNA molecule derived from siRNA metabolic degradation.

6. The method of claim 1, wherein said oligonucleotide template is an RNA molecule.

7. The method of claim 1, wherein said oligonucleotide template is a DNA molecule.

8. The method of claim 1, wherein said oligonucletide template has a spacer sequence of 10 to 1000 bases.

9. The method of claim 1, wherein said oligonucleotide template contains a non-extendable base at its 3′-end.

10. The method of claim 1 wherein said DNA polymerase is a Reverse Trascriptase that synthesizes a complementary DNA strand to an RNA template.

11. The method of claim 1, wherein said DNA polymerase lacks 5′-nuclease activity.

12. The method of claim 1, wherein said amplification regimen comprises a polymerase chain reaction.

13. The method of claim 1, wherein said amplification regimen comprises trascription using an RNA polymerase.

14. The method of claim 2 wherein the step of measuring the quantity is performed using quantitative polymerase chain reaction.

15. The method of claim 2 wherein the step of measuring the quantity is performed using a real-time polymerase chain reaction.

16. The method of claim 1, wherein a plurality of short RNA sequences are detected and/or quantitated in a single reaction.

17. The method of claim 16 wherein a plurality of short RNA sequences are detected and quantified in a single assay using a different oligonucleotide template for each short RNA sequence of said plurality, wherein each different oligonucleotide template has a unique spacer sequence.

18. The method of claim 16 wherein a plurality of short RNA sequences are detected and quantitated using PCR amplification of sequences encoded in respective unique spacer sequences, wherein PCR amplicons are designed to produce amplified products of unique length for each of the short RNAs targeted in the assay.

19. The method of claim 17 wherein:
a) each said template comprises in its spacer sequence a pair of sequences shared by each said spacer in each said template, the members of said pair of sequences separated by a sequence of unique length for each template specific for a different short RNA;
b) PCR amplification is conducted using primers encoding or complementary to the members of said pair; and
c) PCR amplification generates amplified products of the length which is unique for each of the short RNAs targeted in the assay.

20. The method of claim 19 wherein the amplified DNA fragments are separated by size.

21. The method of claim 19 wherein the separation is performed by electrophoresis or chromatography.

22. The method of claim 19 wherein the separation is performed by capillary electrophoresis.

23. The method of claim 14 wherein primers used for said polymerase chain reaction are fluorescently labeled.

24. A method of detecting a short RNA of interest in a nucleic acid sample, wherein said short RNA has a 5′ and a 3′ end, comprising:
a) hybridizing a DNA template to said sample under conditions wherein said template binds to said short RNA, said template having a 3′ and a 5′ end, said template comprising in order from its 3′ end to its 5′ end, a sequence complementary to said short RNA, a spacer of defined sequence and length, and a sequence encoding an RNA polymerase promoter,
b) extending said short RNA using a DNA polymerase to produce an extended short RNA product, such that said extended short RNA product comprises said short RNA and an extension adjacent to the 3′ end of the short RNA, wherein said extension comprises, in order from its 5′ end to its 3′ end, a sequence complementary to said spacer of defined sequence and length, and a sequence complimentary to an RNA polymerase promoter,
c) transcribing the extended product formed in step (b) using an RNA polymerase which recognizes said RNA polymerase promoter, such that an RNA product is formed comprising in order from its 5′ to 3′ end, said spacer of defined length and a sequence complementary to said short RNA; and
d) detecting the RNA product formed in step (c), wherein the detection of the RNA product formed in step c indicates the presence of said short RNA of interest in said nucleic acid sample.

25. The method of claim 24, wherein the transcribing step (c) is performed in the presence of one or more labeled nucleotides, such that said nucleotides are incorporated into said RNA product of step (c),

26. The method of claim 24, wherein said RNA product of step (c) is detected using capillary electrophoresis.

27. The method of claim 24, further comprising separating the RNA product of step (c) by capillary electrophoresis.

28. The method of claim 1, further comprising quantitating the amplified RNA, wherein said quantitating determines an initial quantity of said short RNA.

29. A method of detecting a plurality of suspected short RNAs of interest in a nucleic acid sample, wherein each of said short RNAs has a 5′ and a 3′ end, comprising:
a) hybridizing a plurality of different DNA templates to said sample under conditions wherein each of said plurality of templates specifically binds to one of said plurality of suspected short RNAs, each of said templates having a 3′ and a 5′ end, each of said templates comprising in order from its 3′ end to its 5′ end, a sequence complementary to one of said plurality of suspected short RNAs, a spacer of defined length, and a sequence complementary to an RNA polymerase promoter, wherein the length of said spacer of defined length is different in each of said plurality of primers;
b) extending each of said plurality of suspected short RNAs using a DNA polymerase to produce a plurality of extended short RNA products, respectively, such that said plurality of extended short RNA products each comprises its respective short RNA and an extension adjacent to the 3′ end of the short RNA, wherein said extension comprises, in order from its 5′ end to its 3′ end, a sequence complementary to said spacer of defined length, and a sequence encoding an RNA polymerase promoter;
c) transcribing each of the extended short RNA products formed in step (b) using an RNA polymerase which recognizes said RNA polymerase promoter, such that each RNA product formed comprises in order from its 3′ to 5′ end, its respective sequence of the spacer of defined length and a sequence complementary to its respective short RNA,
d) detecting each of the distinct RNA products formed in step (c), wherein the detection of a plurality of distinct RNA products formed in step (c) indicates the presence of a plurality of said suspected short RNAs of interest in said nucleic acid sample.

30. The method of claim 29, wherein said transcribing step (c) is performed in the presence of one or more labeled nucleotides, such that said nucleotides are incorporated into said RNA product of step c,

31. The method of claim 29, wherein the RNA product of step (c) is detected using capillary electrophoresis.

32. The method of claim 29, further comprising separating the RNA product of step (c) by capillary electrophoresis.

33. The method of claim 29, further comprising quantitating a transcribed RNA product to determine an initial amount of a short RNA.

34. The method of claim 29, further comprising comparing the relative quantity of an RNA product relative to a standard or to another transcribed RNA.
